(19) United States
(12) Patent Application Publication
(10) Pub. No.: US 2016/0067073 A1
(43) Pub. Date: Mar. 10, 2016

(54) WEIGHT LOSS DEVICE

(71) Applicants: Roberta CAROTENUTO, Napoli (IT); Giovanni TARTAGLIA, Napoli (IT)

(72) Inventor: Roberta CAROTENUTO, Napoli (IT)

(21) Appl. No.: 14/868,210

(22) Filed: Sep. 28, 2015

Related U.S. Application Data
(63) Continuation of application No. PCT/IB2013/060247, filed on Nov. 19, 2013.

(30) Foreign Application Priority Data
Mar. 28, 2013 (IT) MI2013A000474

(51) Int. Cl. A61F 5/00; A61C 5/14

(52) U.S. Cl. CPC A61F 5/0006; A61C 5/14

(57) ABSTRACT

A weight loss device including at least one peripheral plate that can be fixed to at least one portion of an upper dental arch or lower dental arch by fixing means, in a fixing configuration. The peripheral plate supports, by way of supporting elements, at least one central plate, which is adapted, in the fixing configuration, to rest against the palate or the floor of the mouth in order to limit the movements of the tongue inside the mouth.

### CROSS REFERENCE TO RELATED APPLICATIONS

This application is a continuation of International Application No. PCT/IB2013/060247, filed on Nov. 19, 2013, which claims priority to Italian Patent Application No. MI2013A000474, filed on Mar. 28, 2013, the entire contents of all of which are incorporated herein by reference in their entirety for all purposes.

### TECHNICAL FIELD

The present invention relates to a weight loss device.

### BACKGROUND

Losing weight and maintaining one's own body weight are aspects that currently and especially in the more industrialized countries affect an increasing number of people.

Currently, the number and variety of diets, in combination or not with physical activity programs, are growing constantly, and so are the number and variety of treatments of the surgical or pharmacological type for weight loss.

However, on the one hand diets and physical activity programs require drastic changes to one's own lifestyle. Due to lack of willpower or also due to practical impossibilities, they are often difficult to practice with constancy and determination. On the other hand, treatments of the pharmacological or surgical type are certainly more invasive, can entail collateral problems and are therefore limited to the more strictly pathological cases of obesity.

### Background

In this context, the aim of the present invention is to provide a weight loss aid device that overcomes the limitations of known types of weight loss method and is particularly effective and immediate.

Within this aim, the present invention provides a weight loss device that is physiologically scarcely invasive and does not entail the risk of important side effects.

The invention further provides a weight loss device that does not require such willpower and commitment as to make its use extremely irregular, with consequent loss of results, for the person who uses it.

The invention also provides a weight loss device that is capable of giving the greatest assurances of reliability and safety in use.

Additionally, the invention provides a weight loss device that is easy to provide and economically competitive with reference to the background art.

More particularly, the invention provides a weight loss device including at least one peripheral plate that can be fixed to at least one portion of a dental arch by fixing means, in a fixing configuration, said at least one peripheral plate supporting, by way of supporting means, at least one central plate, which is adapted, in said fixing configuration, to limit the movements of the tongue inside the mouth.

### BRIEF DESCRIPTION OF THE DRAWINGS

Further characteristics and advantages will become better apparent from the description of two preferred but not exclusive embodiments of a weight loss device, illustrated by way of nonlimiting example with the aid of the accompanying drawings, wherein:

FIG. 1 is a rear perspective view of a first embodiment of a weight loss device according to the invention;

FIG. 2 is a front perspective view of the device of FIG. 1 according to the invention;

FIG. 3 is a bottom plan view of the device of FIG. 1, according to the invention, applied to an upper dental arch;

FIG. 4 is a sectional side view of the device of FIG. 1, according to the invention, applied to an upper dental arch;

FIG. 5 is a top plan view of a second embodiment of a weight loss device, according to the invention, applied to a lower dental arch;

FIG. 6 is a sectional side view of the device of FIG. 5, according to the invention, applied to a lower dental arch;

FIG. 7 is a rear perspective view of a first variation of the first embodiment of the weight loss device shown in FIG. 1, applied to an upper dental arch;

FIG. 8 is a rear perspective view of a second variation of the first embodiment of the weight loss device shown in FIG. 1, applied to an upper dental arch;

FIG. 9 is a bottom plan view of a third variation of the first embodiment of the weight loss device shown in FIG. 1, applied to an upper dental arch;

FIG. 10 is a rear perspective view of a fourth variation of the first embodiment of the weight loss device shown in FIG. 1, applied to an upper dental arch;

FIG. 11 is a rear perspective view of a fifth variation of the first embodiment of the weight loss device shown in FIG. 1, applied to an upper dental arch;

FIG. 12 is a sectional side view of the weight loss device according to the invention, applied to an upper dental arch.

### DETAILED DESCRIPTION

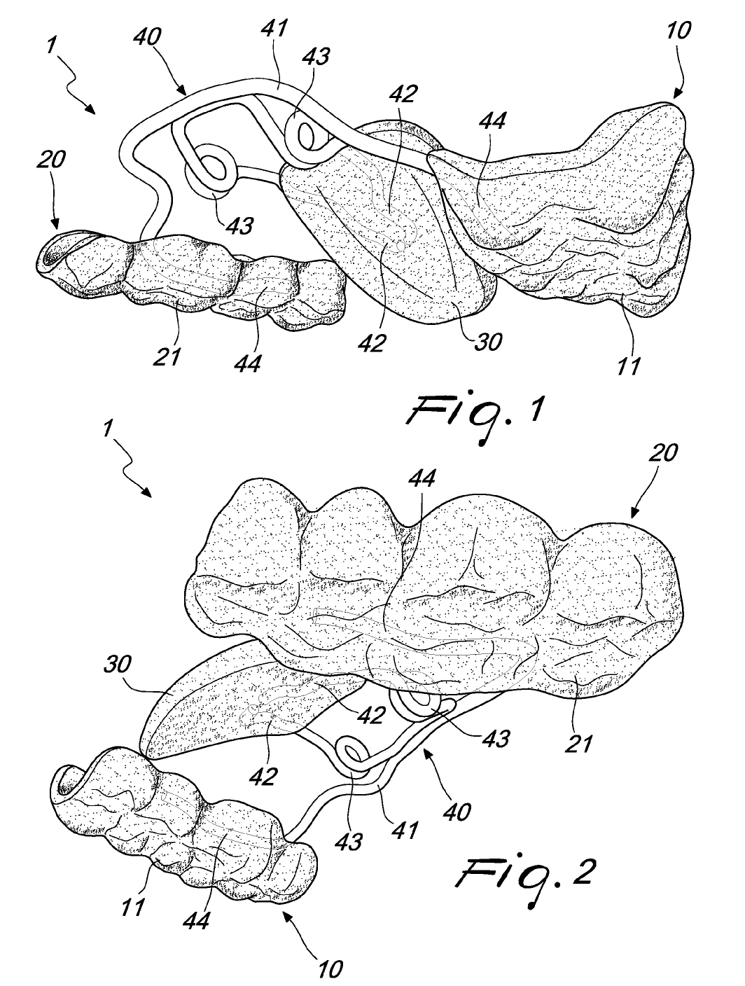

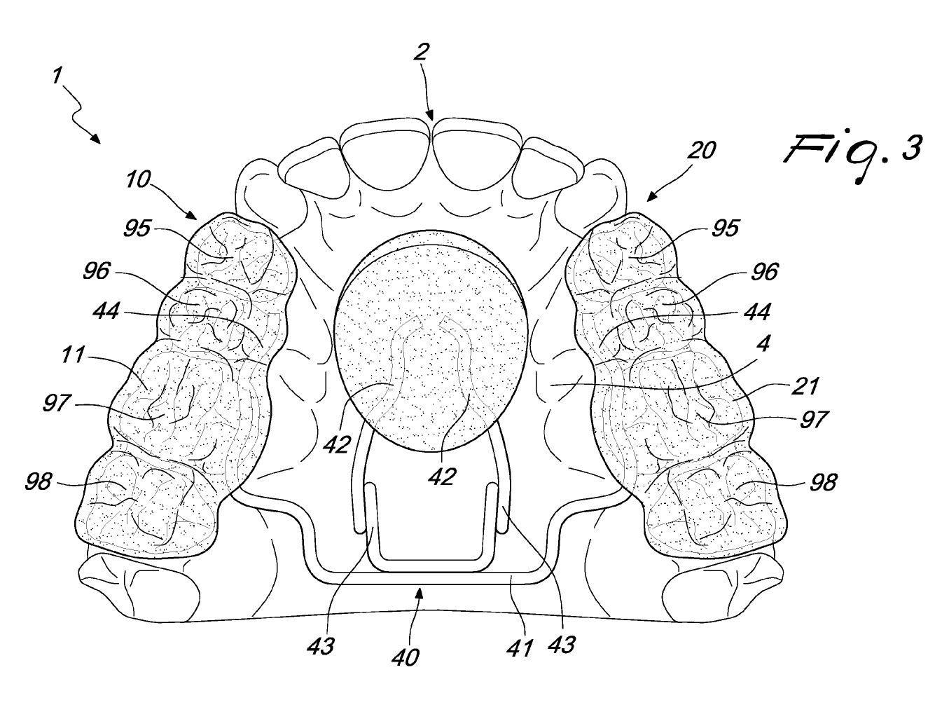

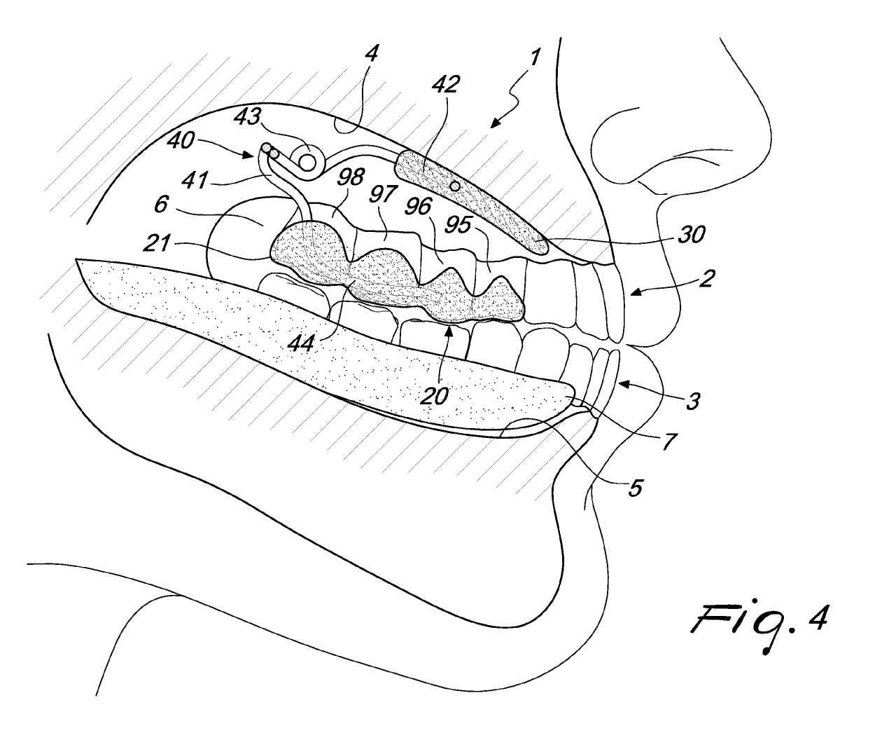

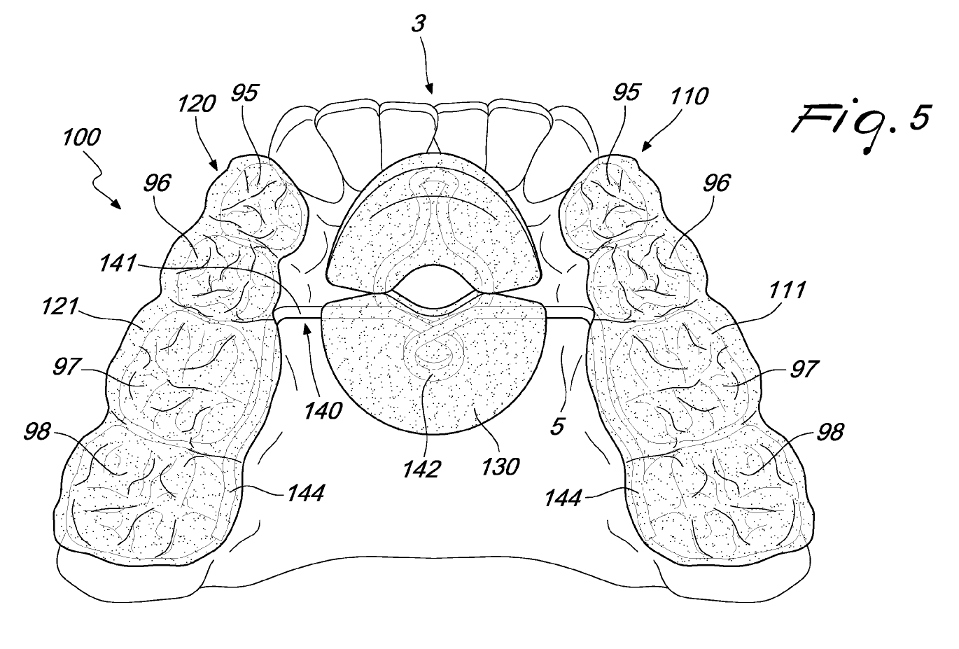

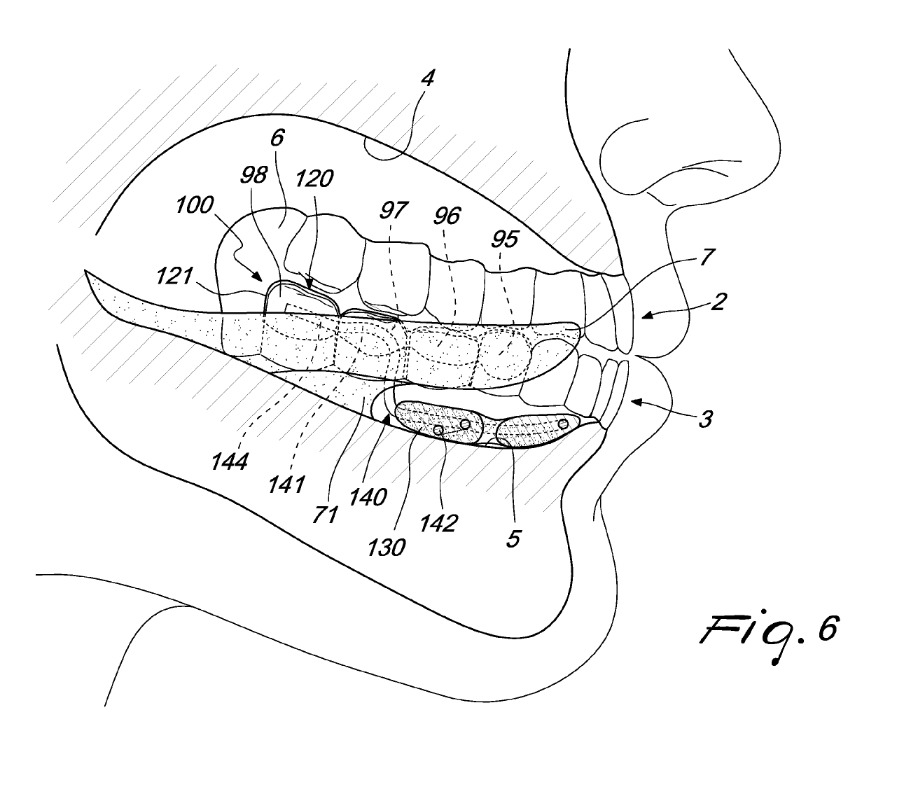

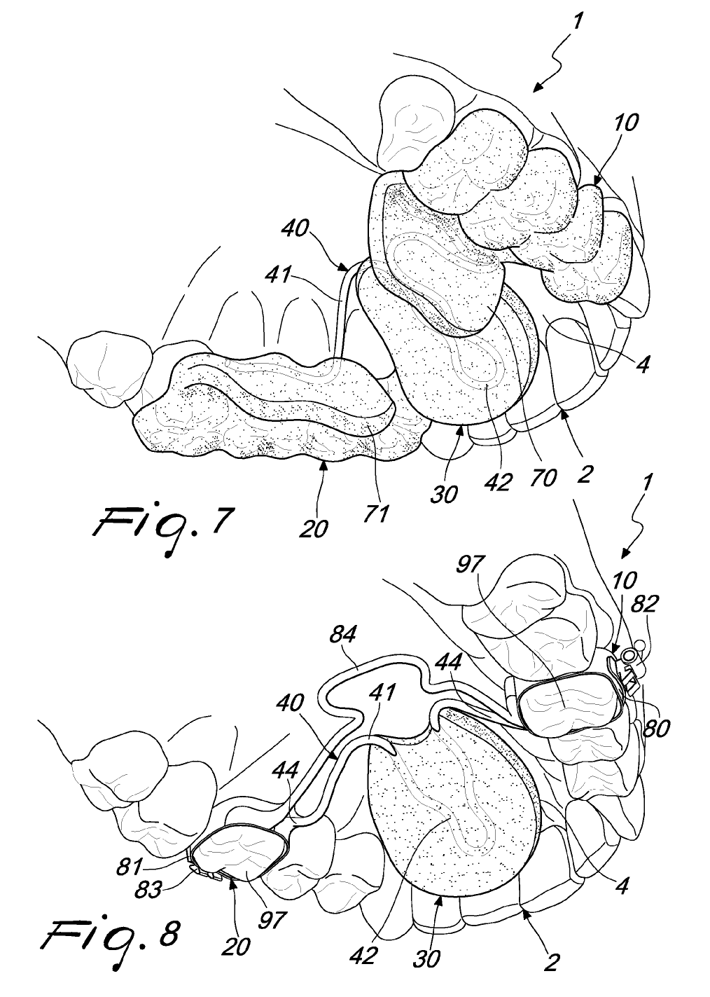

With reference to the figures, the weight loss device is designated generally by the reference numeral 1 in the case of the first embodiment and of its five variations, shown in FIGS. 1 to 4 and in FIGS. 7 to 11, and by the reference numeral 100 in the case of the second embodiment, shown in FIGS. 5 and 6.

According to the invention, the weight loss device 1, 100 comprises at least one peripheral plate 10, 20, 110, 120, which can be fixed to at least one portion of an upper dental arch 2 or lower dental arch 3 by way of fixing means in a so-called fixing configuration.

Such peripheral plate 10, 20, 110, 120 supports, by way of supporting means 40, 140, at least one central plate 30, 130, which is adapted, in the fixing configuration, to rest against the palate 4 or the floor of the mouth 5 in order to limit the movements of the tongue 7 inside the mouth 6.

The weight loss device 1, 100 achieves a weight loss function since it limits the movements of the tongue during chewing, making chewing difficult.

Furthermore, advantageously, the weight loss device 1, 100 is adapted to reduce the useful volume of the mouth 6, particularly due to the presence of the central plate 30, 130. As a consequence of this reduction in useful volume of the mouth 6, chewing is rendered not only difficult but also slower, this contributing further to the weight loss function of said device 1, 100.

Advantageously, said fixing means are selected from the group of adhesives for dentistry applications.

The weight loss device 1, 100 is therefore adapted to be cemented with said adhesives to the upper dental arch 2 or lower dental arch 3 of an individual, so that said individual cannot remove it freely and autonomously. The removal of the weight loss device 1, 100 is in fact possible only by qualified personnel or other specialized personnel of the field, by way of adapted adhesive removal methods that are known in the art.

As an alternative, said fixing means can be selected from the group of temporary adhesives, such as for example adhesive creams for dentures that are available in pharmacies. In this manner, the weight loss device 1, 100 can be applied to the upper dental arch 2 or lower dental arch 3 as well as removed directly and autonomously by the individual himself

As a further alternative, said fixing means can also consist of the interlocking effect of the peripheral plate 10, 20, 110, 120 on the upper dental arch 2 or lower dental arch 3, so that the application of the weight loss device 1 does not require the use of glues and/or temporary adhesives.

On the peripheral plate 10, 20, 110, 120 there is the contour 11, 21, 111, 121 of at least one portion of an upper dental arch 2 or lower dental arch 3 for fixing the peripheral plate 10, 20, 110, 120 to the dental arch portion 2, 3.

The at least one peripheral plate 10, 20, 110, 120 comprises a right peripheral plate 10, 110 and a left peripheral plate 20, 120, which can be fixed respectively to the right and left hemiarches of the upper dental arch 2 or lower dental arch 3.

In a preferred embodiment, the portion of dental arch 2, 3 to which the peripheral plate 10, 20, 110, 120 can be fixed comprises the following teeth: first premolar 95, second premolar 96, first molar 97, second molar 98.

The portion of dental arch 2, 3 to which it is possible to fix the peripheral plate 10, 20, 110, 120 can also comprise the third molar tooth.

The supporting means 40, 140 can comprise a metal wire 41, 141 that is embedded partially in the peripheral plates 10, 20, 110, 120 and in the central plate 30, 130.

According to the first embodiment of the weight loss device 1, shown in FIGS. 1 to 4, the metal wire 41 forms a palate barrier bar that joins the right peripheral plate 10 and the left peripheral plate 20, forming a bridge between the two hemiarches and giving solidity to the weight loss device 1 proper.

At its ends 44, said metal wire 41 is advantageously embedded in the peripheral plates 10 and 20 at, and parallel to, the second premolar tooth 96, the first molar tooth 97 and the second molar tooth 98.

In a central portion with respect to the two peripheral plates 10 and 20, the wire 41 can comprise portions 42 embedded in the central plate 30.

A similar metal wire structure 141 can be present also in the weight loss device 100 according to the second embodiment shown in FIGS. 5 and 6, where the ends 144 of the wire 141 are embedded in the peripheral plates 110 and 120 and wherein the central portion 142 is embedded in the central plate 130.

In this second embodiment, the central plate 130 has a bilobate shape, wherein a central portion 142 of the metal wire 141 is embedded in both lobes. A concave recess can be present between the two lobes.

Furthermore, with particular reference to the first embodiment of the invention, proximate to the portions 42 of metal wire 41 that are embedded in the central plate 30 the metal were 41 can comprise helical portions 43, which are adapted to apply such an elastic force as to keep the central plate 30 rested against the palate 4, depending on the different palate shapes that each individual may have.

The weight loss device 1, 100 can comprise, in the central plate 30, 130, at least one medicalized portion adapted to be impregnated or filled with anorectic substances, preferably natural anorectic substances.

The central plate 30, 130 advantageously has a substantially ovoid or disk-like plan shape and is arranged during use preferably adjacent to the medial incisors. Advantageously, the central plate 30, 130 can also arrange itself in a rearward position with respect to the medial incisors, toward the back of the mouth 6.

The shape of the central plate 30, 130 is furthermore slightly concave in the part that faces the tongue 7, so that the tongue 7 is trapped in said concavity and undergoes a reduction in the movements that are allowed to it within the mouth 6.

In a particularly effective embodiment, the central plate 30 of the weight loss device 1 for the upper dental arch 2 has an inclination of approximately 30° downwardly by with respect to a transverse plane that passes through the mouth 6, so that the central plate 30 follows the profile of the palate 4 in the sagittal plane.

Likewise, the central plate 130 of the weight loss device 100 for the lower dental arch 3 preferably has an inclination of approximately 30° upwardly with respect to a transverse plane that passes through the mouth 6, so that the central plate 130 follows the profile of the floor of the mouth 5 in the sagittal plane.

During use, the central plate 130, in the case of the weight loss device 100 for the lower dental arch 3, is arranged in front of the frenulum of the tongue 71.

The weight loss device 1, 100 comprises advantageously a first weight loss device 1 for the upper dental arch 2 and a second weight loss device 100 for the lower dental arch 3. In other words, it is possible to apply advantageously in an individual the weight loss device 1 for the upper arch 2, or the weight loss device 100 for the lower arch 3, or, preferably, both weight loss devices 1 and 100.

The weight loss device 1, 100 can be made advantageously of a material based on resin or silicone, wherein it is possible to use light metals or light metal alloys for the metal wires 41, 141.

The weight loss device 1, 100 is orthodontically inert, i.e., it does not have the function of moving the teeth, to which it is simply fixed.

FIG. 7 illustrates a first variation of the weight loss device 1. According to this variation, the peripheral plates 10, 20 each comprise at least one wing 70, 71 that protrudes toward the inside of the mouth 6. The presence of said protruding wings 70, 71 is adapted to reduce further the useful volume of the mouth 6 and to limit further the movements of the tongue 7, making chewing even more difficult and time-consuming The protruding wings 70, 71 are advantageously made of the same material of which the peripheral plates 10, 20 are made and are preferably provided as extensions of said peripheral plates 10, 20. The weight loss device 100 for the lower dental arch 3, according to the second embodiment, also can comprise said protruding wings.

Advantageously, the central plate 30, 130 also can comprise one or more wings that protrude toward the inside of the mouth 6 and are optionally provided as extensions of said central plate 30, 130.

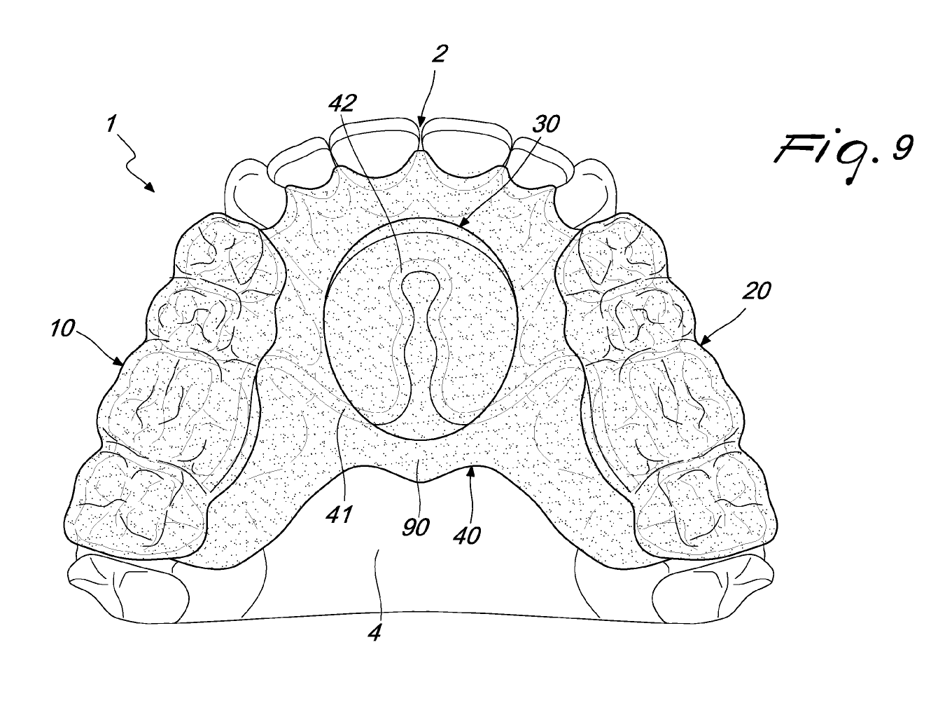

FIG. 8 illustrates a second variation of the weight loss device 1. According to this variation, the peripheral plates 10, 20 are in the form of a pair of metal bands 80, 81, each of which surrounds at least one tooth of the dental arch 2. In FIG. 8, the metal bands 80 and 81 are applied to the first molar teeth 97. Said metal bands 80, 81 can be fixed to the respective tooth by way of dental adhesives and/or locking means 82, 83 of the screw type. Advantageously, the metal wire 41 extends, at its ends 44, from the metal bands 80 and 81, so that its central portion 42 is embedded in the central plate 30. In addition to the metal wire 41, the device 1 can also comprise an additional metal wire 84 that is adapted to join directly the two metal bands 80 and 81, forming a second bridge between the two hemiarches and giving additional solidity to the weight loss device 1. The weight loss device 100 for the lower dental arch 3, according to the second embodiment, also can comprise said metal bands that surround at least one pair of opposite teeth of the lower dental arch.

FIG. 9 shows a third variation of the weight loss device 1. According to this variation, the weight loss device 1 comprises a medial plate 90 that is adapted to adhere to the palate 4 or to the floor of the mouth 5 in the case of a weight loss device 100 for a lower dental arch 3. The medial plate 90 is arranged between the palate 4 or the floor of the mouth 5 and the central plate 30, 130. Said medial plate 90 is advantageously joined to the peripheral plates 10 and 20 so as to form a monolithic body. Said medial plate 90 can advantageously be extended along the entire palate surface that is inscribed in the upper dental arch 2, until it is adjacent to the teeth. Advantageously, moreover, the metal wire 41 can be embedded completely in the medial plate 90. The third variation of weight loss device 1 shown in FIG. 9 is particularly adapted to be applied to the dental arch 2 by means of temporary adhesives, such as adhesive creams for dentures, in view of the abundant bonding surface that is available. The medial plate 90 is made advantageously of the same material of which the peripheral plates 10, 20 are made, preferably starting from a same mold.

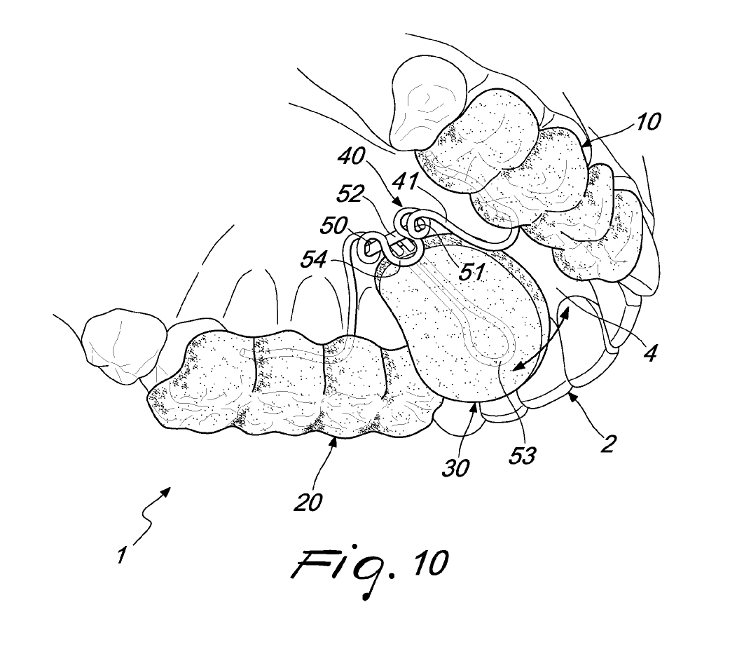

FIG. 10 illustrates a fourth variation of the weight loss device 1. According to this variation, the central plate 30 of the weight loss device 1 can tilt. In particular, said central plate 30 advantageously is pivoted to the supporting means 40 so that it can oscillate from a position for resting against the palate 4 to a position for spacing from the palate 4. In this manner, the central plate 30 tends, due to gravity, to tilt in the position for spacing from the palate 4. In said position for spacing from the palate 4, the tilting central plate 30 reduces further, with its space occupation, the useful volume of the mouth 6 and limits further the movements of the tongue 7, making chewing even more difficult and time-consuming Advantageously, the metal wire 41 is provided so as to form two slots 50 and 51, inside which a pivot 52 can rotate freely and is integral with a metal wire 53 that is embedded in the central plate 30. Advantageously, furthermore, in its central portion, the metal wire 41 also provides an abutment element 54 that limits the downwardly oscillation of the central plate 30. The inclination of the tilting central plate 30 with respect to the plane defined by the palate 4 is in fact comprised in the interval between 0 and 30°.

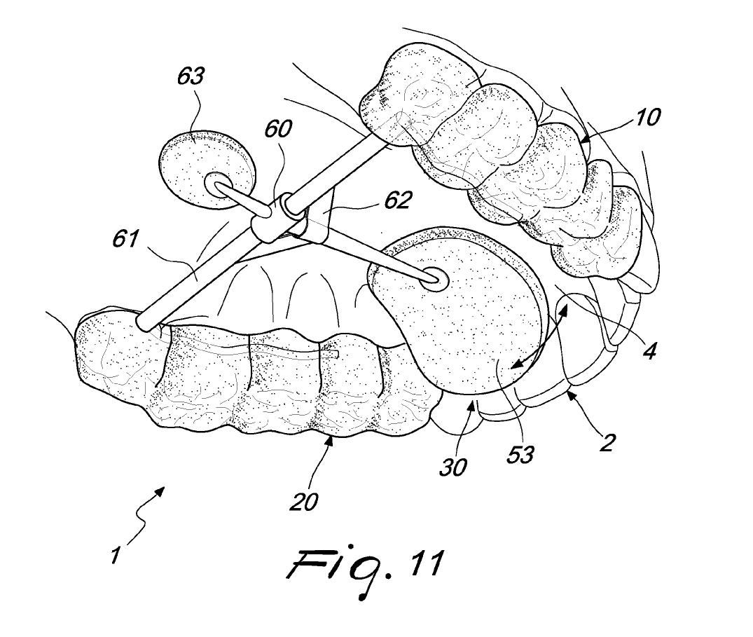

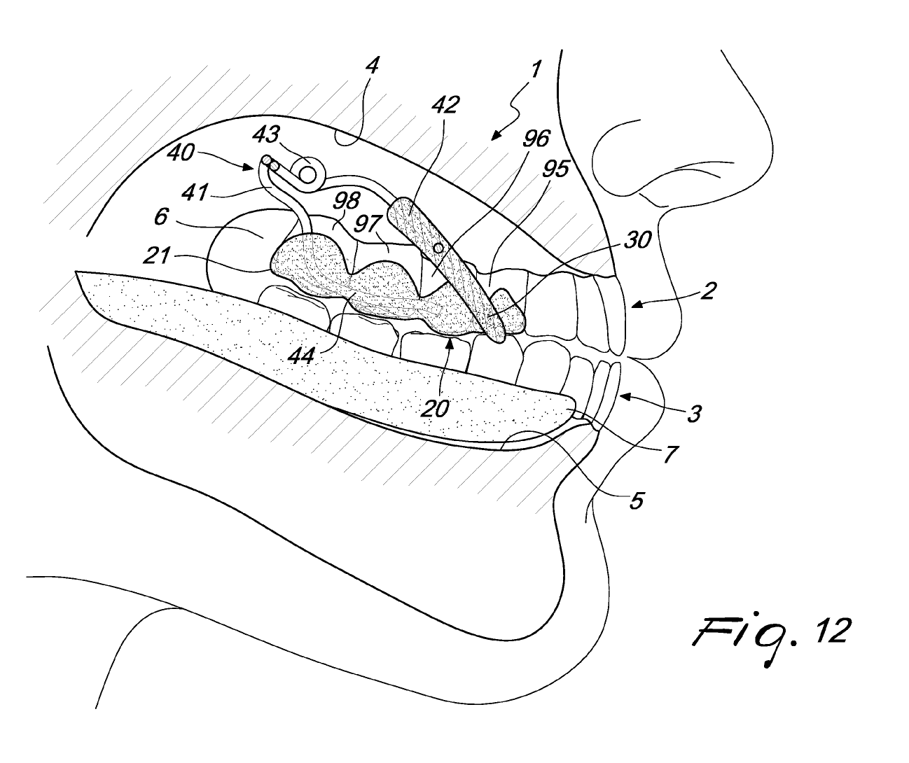

FIG. 11 illustrates a fifth variation of the weight loss device 1. According to this variation, and in a manner similar to the fourth variation, the central plate 30 of the weight loss device 1 can tilt. In particular, said central plate 30 comprises a sleeve 60, which can rotate with respect to a pivot 61 that passes through it and joins in a bridge-like manner the two peripheral plates 10 and 20. In this manner, the peripheral plate 30 can perform an oscillation from a position for resting against the palate 4 to a position for separation from the palate 4.

Advantageously, the pivot 60 comprises in its central portion, an extended abutment portion 62 that limits the downwardly oscillation of the central plate 30.

Advantageously, moreover, the weight loss device 1 can comprise a small rear plate 63, which is integral with the sleeve 61 and, by resting against the palate 4, can limit the downwardly oscillation of the central plate 30.

The weight loss device 100 for the lower dental arch 3, according to the second embodiment, also can comprise a tilting central plate, shown with reference to the fourth and fifth variations of the weight loss device 1, although due to gravity it tends to remain resting against the floor of the mouth 5.

Advantageously, furthermore, the peripheral plates 10, 20, 110 and 120 can also be extended to the entire upper dental arch 2 or lower dental arch 3, therefore covering all the teeth.

The technical characteristics shown and described in relation to the two embodiments and to the related five variations of the weight loss device 1, 100 can be combined and integrated variously in a single weight loss device 1, 100.

Preferably, moreover, as shown by way of example in FIG. 12 for the device 1, in all the embodiments of the weight loss device 1, 100 and in all the corresponding variations the central plate 30, 130 is adapted to protrude within the mouth 6, arranging itself within the mouth 6 in a position that is intermediate between the palate 4 and the floor of the mouth 5, in order to limit the freedom of motion of the tongue 7 and reduce the volume of the mouth 6.

In particular, the central plate 30, 130 can arrange itself in a position of full separation from the palate 4 and/or from the floor of the mouth 5.

Advantageously, moreover, the central plate 30, 130 can arrange itself substantially at the center of the mouth 6 and preferably in a central position that is substantially equidistant from the palate 4 and from the floor of the mouth 5.

The provision of the weight loss device 1, 100 provides for the steps of taking the impression of one or both dental arches 2, 3 and of obtaining from the cast thereof the structure of the peripheral plates 10, 20, 100, 120 of the weight loss device 1, 100, which is then assembled with the supporting means 40, 140 and with the central plate 30, 130.

It is further possible to provide in the structure a portion that can be impregnated or an adapted recess, into which anorectic gel of a natural type can be applied.

Moreover, in order to limit the possibility of discomfort or temporary dysfunctions of the tongue, the use of the weight loss device can be divided into cycles. For example, it is possible to practice cycles of use of the weight loss device 1, 100 that last twenty days, with one-week rest intervals, while performing exercises for tongue posture.

Operation of the weight loss device 1, 100 is clear and evident from what has been described above: its presence in the mouth 6 limits the movements of the tongue 7, making chewing more difficult. Moreover, since it cannot be removed freely and arbitrarily by the individual who wears it, but only at qualified personnel, the individual is forced to wear it continually.

The application of the weight loss device 1, 100 with temporary adhesives is provided in the test periods that precede the final application with adhesives for dentistry applications, or in said rest cycles, in order to leave greater freedom of management of the therapy to the individual. Furthermore, the application of the weight loss device 1, 100 with temporary adhesives can be provided for maintenance treatments, at the end of the period of treatment with the device applied permanently with adhesives for dental applications, when the individual has reestablished his own dietary balance but still requires an extension of therapy, albeit with greater freedom of management thereof

In practice it has been found that the weight loss device according to the present invention achieves the intended aim, since it allows to overcome the limitations of weight loss methods of the known type, in a particularly effective and immediate manner.

In particular, while keeping oral functionalities intact, it introduces a relative discomfort in chewing, increasing the time required to eat and consequently also decreasing the impulse to eat more food than necessary.

Another advantage of the weight loss device according to the invention is that it is physiologically scarcely invasive and does not entail side effects where it is installed and used appropriately and as described above.

A further advantage of the weight loss device according to the invention is that it can be used regularly and continually regardless of the temporary and variable will of the individual but who wishes to lose weight or must lose weight.

Another advantage of the weight loss device according to the invention resides in that it has no medical contraindications and in that there are no pathologies that may make its use not advisable or even prevent its use, as instead occurs in many of the known types of weight loss method.

A further advantage of the weight loss device according to the invention resides in that for the entire period of use the subject is not forced to follow unbalanced diets but can follow a balanced diet of the Mediterranean type. Furthermore, the choice of foods is “forced”, by the presence of the device itself, toward food that is simpler and less complicated, indeed due to the difficulty in chewing and swallowing.

The weight loss device thus conceived is susceptible of numerous modifications and variations, all of which are within the scope of the accompanying claims.

All the details may further be replaced with other technically equivalent elements.

In practice, the materials used, so long as they are compatible with the specific use, as well as the contingent shapes and dimensions, may be any according to requirements.

The disclosures in Italian Patent Application No. MI2013A000474, from which this application claims priority, are incorporated herein by reference.

1-17. (canceled)

18. A weight loss device, comprising at least one peripheral plate that can be fixed to at least one portion of a dental arch by fixing means, in a fixing configuration, said at least one peripheral plate supporting, by way of supporting means, at least one central plate, which is adapted, in said fixing configuration, to limit movements of the tongue inside the mouth.

19. The weight loss device according to claim 18, wherein said dental arch is an upper dental arch and said at least one central plate is shaped, in said fixing configuration, in order to rest against the palate.

20. The weight loss device according to claim 19, wherein said dental arch is a lower dental arch and said at least one central plate is shaped, in said fixing configuration, so as to rest against the floor of the mouth.

21. The weight loss device according to claim 18, wherein said fixing means are selected from the group of adhesives for dentistry applications.

22. The weight loss device according to claim 18, wherein a contour of at least one portion of an upper dental arch or lower dental arch is provided on said at least one peripheral plate for the fixing of said at least one peripheral plate to said dental arch portion.

23. The weight loss device according to claim 22, wherein said at least one peripheral plate comprises a right peripheral plate and a left peripheral plate that can be fixed respectively to left and right hemiarches of said dental arch.

24. The weight loss device according to claim 23, wherein said portion of dental arch to which said peripheral plate can be fixed comprises the first premolar, second premolar, first molar and second molar teeth.

25. The weight loss device according to claim 18, wherein said supporting means comprise a metallic wire that is partially embedded in said at least one peripheral plate and in said central plate.

26. The weight loss device according to claim 18, wherein said central plate comprises at least one medicalized portion that is adapted to be impregnated or filled with anorectic substances.

27. The weight loss device according to claim 18, wherein said central plate is arranged adjacent to medial incisors.

28. The weight loss device according to claim 18, wherein said fixing means are selected from the group of temporary adhesives.

29. The weight loss device according to claim 18, wherein said at least one peripheral plate comprises at least one wing that protrudes toward the inside of said mouth.

30. The weight loss device according to claim 19, wherein said at least one peripheral plate is in the form of a metal band that surrounds at least one tooth of said dental arch.

31. The weight loss device according to claim 18, further comprising a medial plate that is adapted to adhere to a palate or to the floor of the mouth and is arranged between the palate or the floor of the mouth and said central plate.

32. The weight loss device according to claim 31, wherein said medial plate is joined to said peripheral plates so as to form a single body with them.

33. The weight loss device according to claim 18, wherein said central plate can tilt.

34. The weight loss device according to claim 18, wherein said central plate is adapted to arrange itself within said mouth in an intermediate position between the palate and the floor of the mouth, being spaced from said palate and from said floor of the mouth.
